(19) United States
(12) Patent Application Publication
(10) Pub. No.: US 2005/0233345 A1
(43) Pub. Date: Oct. 20, 2005

(54) Primers for detecting food poisoning bacteria and a use thereof

(76) Inventors: Banda Padmanabha Padmapriya, Karnataka (IN); Aiyagari Ramesh, Karnataka (IN); Arun Chandrashekar, Karnataka (IN); Mandyam Chakravarathy Varadaraj, Karnataka (IN)

Correspondence Address: Thomas J. Kowalski, Esq.;c/o FROMMER LAWRENCE & HAUG, LLP, 745 Fifth Avenue, New York, NY, 10151, US

(21) Appl. No.: 10/951,225

(22) Filed: Sep. 27, 2004

Related U.S. Application Data
(63) Continuation-in-part of application No. PCT/IB02/01150, filed on Mar. 26, 2002.

(30) Foreign Application Priority Data
Oct. 2, 2003 (WO) 03/080865

(51) Int. Cl. C12Q 1/68; G06F 19/00; G01N 33/48; G01N 33/50; C07H 21/04

(52) U.S. Cl. 435/6; 536/24.1; 702/20

(57) ABSTRACT

Provided are novel primers directed against enterotoxin A gene (ent A) of bacteria Staphylococcus aureus and primers directed against heat stable enterotoxin gene (yst) of bacteria Yersinia enterocolitica, for detecting poisoning in food articles. Also provided is a highly sensitive method for detecting bacterial food poisoning using the primers.

### REFERENCE TO RELATED APPLICATIONS

This application is a continuation-in-part of International Application PCT/IB02/01150 filed on Mar. 26, 2002 and published as WO 03/080865 on Oct. 2, 2003.

The foregoing application, and each document cited or referenced in the foregoing application, and any manufacturer's instructions or catalogues for any products cited or mentioned in each of the foregoing applications are incorporated by reference into this application. Documents incorporated by reference into this text or any teachings therein may be used in the practice of this invention. Documents incorporated by reference into this text are not admitted to be prior art.

It is noted that in this disclosure and particularly in the claims, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

### TECHNICAL FIELD

The present invention relates to novel primers of SEQ ID Nos. 1-4 useful for detecting poisoning in food articles wherein primers of SEQ ID Nos. 1 and 2 are directed against enterotoxin A gene (ent A) of bacteria Staphylococcus aureus and primers of SEQ ID Nos. 3 and 4 are directed against heat stable enterotoxin gene (yst) of bacteria yersinia enterocolitica, and a highly sensitive use of detecting said food poisoning bacterial species using said primers.

### BACKGROUND ART

Staphylococcus aureus has long been considered as one of the most important food poisoning bacterial species from the public health point of view. It is ubiquitous in nature, being both a human and a zoonotic commensal (Tamarapu et al. 2001). It is known to produce thermostable enterotoxins causing staphylococcal food poisoning (McLauchlin et al. 2000). Conventionally, Staphylococcus aureus is detected by its ability to reduce tellurite or ferment mannitol in the selective media, followed by the morphological, cultural and biochemical characteristics. (Duguid, 1996).

Among the staphylococcal enterotoxins, enterotoxin A (SEA) is predominantly associated with food poisoning outbreaks. SEA has super antigenic activity as well as enterotoxigenic making itself the most important toxin in the fields of clinical and food microbiology. The nucleotide sequence of the gene encoding enterotoxin A (entA) has been determined and also shown considerable sequence divergence within the family of enterotoxins (Betley and Mekalanos, 1988).

Another significant food poisoning bacterial species from the public health point of view is Yersinia enterocolitica. Strains of Yersinia enterocolitica is an enteroinvasive pathogen prevalent in soil, water and clinical sources. This bacterium is able to survive in both, vacuum packed and refrigerated food samples. Virulence in Yersinia enterocolitica results from a series of plasmid-borne and chromosomally-encoded genetic traits such as the outer membrane proteins and low molecular weight heat stable enterotoxins (Gemski et al. 1990; Ibrahim et al. 1997). The chromosomal heat stable enterotoxin (yst) gene is associated with virulent serotypes of Yersinia enterocolitica and hence, is a useful diagnostic marker (Ibrahim et al. 1992). Conventional methods have been proposed to isolate Yersinia enterocolitica from food samples based on cold enrichment, plating on selective media and characteristic bull's eye colonies (DeBoet and Seldam, 1987).

Advances made in detection system over the years with the availability of the nucleotide sequences has set a path in the application of polymerase chain reaction (PCR) for the detection of specific genes in Staphylococcus aureus and Yersinia enterocolitica in pure culture and food systems using gene specific sets of primers.

Reference may be made to the work of Johnson et al. (1991), who designed the primers internal to the coding region for the toxin gene and could achieve a sensitivity of 10 pg when enterotoxin A set of primers were used. Primers designed for enterotoxin A spanned regions between 490 to 509 for the forward primer and 591 to 610 for the reverse primer based on the gene sequence of Betley and Mekalanos (1988). However, sensitivity of the primers was evaluated only in pure culture and its application in food system was not demonstrated. Moreover, the DNA isolation protocol was cumbersome and included steps of enzymatic treatment and method of phenol: chloroform extraction.

Reference may be made to the work of Tsen et al. (1992) who designed primers for enterotoxin A by comparing sequences of other enterotoxin genes and selecting those regions with least homology. A sensitivity of 1 to 10 cells was achieved in milk and beef samples, respectively. Template DNA preparation employed by the authors was laborious involving the use of specialized enzymes like proteinase K and lysostaphin, followed by phenol: chloroform extraction.

Reference may be made to the work of McLauchlin et al. (2000), who used enterotoxin A specific primers similar to Johnson et al. (1991). However, the investigation was primarily concerned with epidemiological screening of Staphylococcus aureus isolates and the level of sensitivity achieved in food samples was very poor.

Reference may be made to the work of Atanassova et al. (2001), who used primers to amplify enterotoxin A fragment from Staphylococcus aureus. The sequence of the primers used was similar to Johnson et al. (1991). The primers were essentially used to study the prevalence of enterotoxigenic Staphylococcus aureus in raw pork and uncooked smoked ham. Samples were enriched and the DNA isolation protocol was lengthy and laborious. No trials were made to determine the sensitivity of the primers.

Reference may be made to the work of Ibrahim et al. (1992), who designed primers to amplify the enterotoxin (yst) gene of pathogenic Yersinia enterocolitica strains belonging to European and American serovars. However, the investigation was primarily aimed at using PCR as an epidemiological tool to differentiate between two clusters of pathogenic Yersinia enterocolitica strains. The sensitivity of the primers and their potential to detect Yersinia enterocolitica in food systems was not tested.

Reference may be made to the work of Ibrahim et al. (1997), wherein primers were designed based on the sequence of yst gene of Yersinia enterocolitica W 1024. The sensitivity reported for these primers with pure culture of Yersinia enterocolitica 0:3 was 10² CFU. Application of these primers to detect Yersinia enterocolitica in food system was not attempted.

Reference may be made to the work of Vishnubhatla et al. (2001), wherein yst gene specific primers were used in a fluorogenic 5′ nuclease PCR assay. A detection limit of 10² CFU/ml and 10³ CFU/g was achieved in pure cultures and spiked ground pork, respectively. However, the detection time was prolonged by incorporating an enrichment step.

A few patents (U.S. Pat. No. 5654144, U.S. Pat. No. 5846783 and others) have appeared wherein the sequences refer to 16s and 23s ribosomal RNA (Ribo Nucleic Acid) and attachment invasion locus (ail) specific primers used for the detection of Yersinia enterocolitica including pathogenic strains. However, the patent search has shown the absence of any patents for primers specific to enterotoxin A gene in Staphylococcus aureus and heat stable enterotoxin gene in Yersinia enterocolitica.

The drawback of all these methods have been lack of consistency, reproducibility and sensitivity in the detection of enterotoxigenic strains of Staphylococcus aureus and Yersinia enterocolitica. Besides, the methods are cumbersome and involves lengthy procedures of enrichment and treatment with complex enzymes. In most of the methods a step of enrichment in a suitable laboratory growth medium is included which may take 8 to 15 hours of incubation or a week's time in case of Yersinia enterocolitica for building up of cell numbers which can result in target DNA for use in PCR detection. On the contrary, the present invention enables direct detection of Staphylococcus aureus and Yersinia enterocolitica in the food system without any enrichment step(s).

### OBJECTS OF THE PRESENT INVENTION

The main object of the present invention is to develop oligonucleotide primers for detecting pathogenic bacteria Staphylococcus aureus.

Another main object of the present invention is to develop oligonucleotide primers for detecting pathogenic and heat stable bacteria yersinia enterocolitica.

Yet another object of the present invention is to develop a highly sensitive and quick use of detecting food poisoning bacteria Staphylococcus aureus and yersinia enterocolitica.

Still another object of the present invention is to develop a use of preparing primers of SEQ ID Nos. 1-4.

### SUMMARY OF THE PRESENT INVENTION

The present invention relates to novel primers of SEQ ID Nos. 1-4 useful for detecting poisoning in food articles wherein primers of SEQ ID Nos. 1 and 2 are directed against enterotoxin A gene (ent A) of bacteria Staphylococcus aureus and primers of SEQ ID Nos. 3 and 4 are directed against heat stable enterotoxin gene (yst) of bacteria yersinia enterocolitica, and a highly sensitive use of detecting said food poisoning bacterial species using said primers.

### DESCRIPTION OF THE INVENTION

Accordingly, the present invention relates to novel primers of SEQ ID Nos. 1-4 useful for detecting poisoning in food articles wherein primers of SEQ ID Nos. 1 and 2 are directed against enterotoxin A gene (ent A) of bacteria Staphylococcus aureus and primers of SEQ ID Nos. 3 and 4 are directed against heat stable enterotoxin gene (yst) of bacteria yersinia enterocolitica, and a highly sensitive use of detecting said food poisoning bacterial species using said primers.

In one embodiment of the present invention, oligonucleotide primers of SEQ ID Nos. 1, 2, 3, and 4.

In another embodiment of the present invention, wherein said primers are of size 20 nucleotides.

In yet another embodiment of the present invention, wherein primers of SEQ ID Nos. 1, and 2 target enterotoxin A gene (entA) of food poisoning bacterial species Staphylococcus aureus.

In still another embodiment of the present invention, wherein primers of SEQ ID Nos. 3, and 4 target heat stable enterotoxin gene (yst) of Yersinia enterocolitica.

In still another embodiment of the present invention, wherein primer of SEQ ID Nos. 1 and 3 are forward primers.

In still another embodiment of the present invention, wherein primer of SEQ ID No. 2 and 4 are reverse primers.

In further embodiment of the present invention, a use of preparing primers of SEQ ID Nos. 1-4.

In another embodiment of the present invention, identifying conserved sequence of entA, and yst genes of bacterial strains Staphylococcus aureus and Yersinia enterocolitica respectively.

In yet another embodiment of the present invention, generating primers using software programme.

In still another embodiment of the present invention, wherein conserved sequence of entA gene is located in a region between 70-370.

In still another embodiment of the present invention, wherein conserved sequence of yst gene is located in a region between 37-195.

In still another embodiment of the present invention, wherein software programme is Primer 3.0

In further embodiment of the present invention, A highly sensitive and quick use of detecting food poisoning bacterial species staphylococcus aureus and/or Yersinia enterocolitica in food systems using specific primers of SEQ ID Nos. 1 and 2, and/or 3 and 4.

In another embodiment of the present invention, preparing food matrix.

In yet another embodiment of the present invention, extracting total microbial DNA.

In still another embodiment of the present invention, amplifying profile of target gene by PCR using said primers.

In still another embodiment of the present invention, analyzing PCR product by gel-electrophoresis.

In still another embodiment of the present invention, detecting said bacterial strain.

In still another embodiment of the present invention, wherein food system is selected from a group comprising milk, fruit juices, and ice creams.

In still another embodiment of the present invention, wherein extracting DNA by using extraction mixture comprising diethyl ether, chloroform, urea, and sodium dodecyl sulphate (SDS).

In still another embodiment of the present invention, wherein diethyl ether and chloroform are in the ratio ranging between 1:1-1:5.

In still another embodiment of the present invention, wherein concentration of urea is ranging between 1.0 to 4.5 M.

In still another embodiment of the present invention, wherein concentration of SDS is ranging between 0.3-3.0%.

In still another embodiment of the present invention, wherein PCR reaction mixture is comprising Tris Hydrochloric acid (Tris HCl) ranging between 6-15 mM, Potassium Chloride (KCl) ranging between 40-60 mM, Magnesium Chloride (MgCl₂) ranging between 0.3-5.0 mM, gelatin ranging between 0.002-0.05%, individual deoxynucleotide triphosphates ranging between 100-500 μM, each specific primer of claim 1, Taq DNA polymerase ranging between 0.3-5.0 units, template DNA ranging between 0.02-3.0%.

In still another embodiment of the present invention, wherein denaturing DNA in PCR at temperature ranging between 90-98° C. for time period ranging between 1-10 minutes.

In still another embodiment of the present invention, wherein denaturing DNA in PCR at temperature preferably ranging between 93-95° C. for time period ranging between 4-6 minutes.

In still another embodiment of the present invention, wherein running PCR with amplification cycles ranging between 25-45 cycles.

In still another embodiment of the present invention, wherein running PCR with amplification cycles preferably ranging between 32-38 cycles.

In still another embodiment of the present invention, wherein denaturation temperature at each cycle is ranging between 90-98° C. for time period ranging between 30-80 seconds.

In still another embodiment of the present invention, wherein denaturation temperature at each cycle is preferably ranging between 93-95° C. for time period ranging between 55-65 seconds.

In still another embodiment of the present invention, wherein annealing DNA in PCR at temperature ranging between 40-65° C. for time period ranging between 30-90 seconds.

In still another embodiment of the present invention, wherein annealing DNA in PCR at temperature preferably ranging between 53-56° C. for time period ranging between 55-65 seconds.

In still another embodiment of the present invention, wherein extension at PCR is at temperature ranging between 68-76° C. for time period ranging between 40-80 seconds.

In still another embodiment of the present invention, wherein extension at PCR is at temperature preferably ranging between 70-74° C. for time period ranging between 55-65 seconds.

In still another embodiment of the present invention, wherein final extension at PCR is at temperature ranging between 68-76° C. for time period ranging between 2-15 minutes.

In still another embodiment of the present invention, wherein final extension at PCR is at temperature preferably ranging between 55-65° C. for time period ranging between 6-10 minutes.

In still another embodiment of the present invention, wherein gel electrophoresis is run on agarose gel.

In still another embodiment of the present invention, wherein concentration of agarose gel is ranging between 1.0-2.0%.

In still another embodiment of the present invention, wherein staining agarose gel with Ethidium bromide at a concentration ranging between 0.2-1.0 μg/ml.

In still another embodiment of the present invention, wherein stained gel is observed under UV transilluminator.

In still another embodiment of the present invention, wherein said use is used to detect said bacterial strains in quantity as low as one cell.

In still another embodiment of the present invention, wherein said use help prevent food poisoning outbreak.

In still another embodiment of the present invention, wherein said use is a direct use of detecting bacterial strain.

### BRIEF DESCRIPTION OF THE ACCOMPANYING DRAWINGS

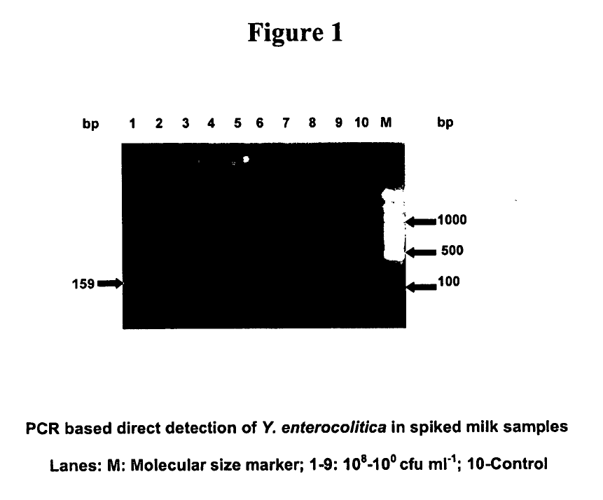

1. FIG. 1 shows PCR based direct detection of Y. enterocolitica in spiked milk samples.

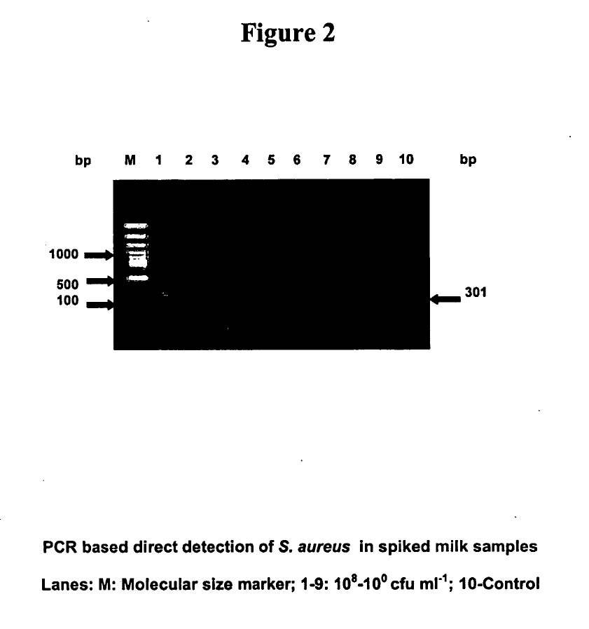

2. FIG. 2 shows PCR based direct detection of S. aureus in spiked milk samples.

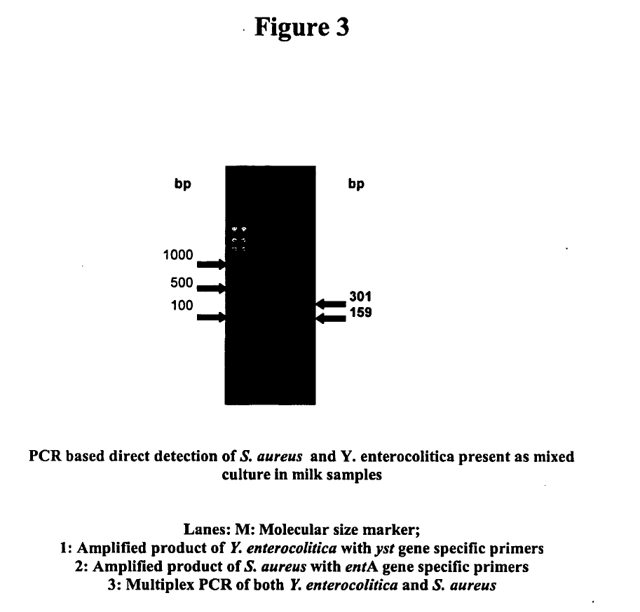

3. FIG. 3 shows PCR based direct detection of Y. enterocolitica and S. aureus present as mixed culture in spiked milk samples.

Further, the present invention provides an improved use for the detection of Staphylococcus aureus (Please refer FIG. 2) and Yersinia enterocolitica (Please refer FIG. 1) in foods which comprises:

(a) designing a set of novel oligonucleotide multiple primers comprising:

| | |
|---|---|
| (i) entA - 1 (F) | |
| 5′ GGTAGCGAGAAAAGCGAAGA 3′ | (SEQ ID NO. 1) |
| and | |
| entA - 2 (R) | |
| 5′ TACCACCCGCACATTGATAA 3′ | (SEQ ID NO. 2) |
| for detecting enterotoxin A target | |
| gene in Staphylococcus aureus, | |
| (ii) yst - 1 (F) | |
| 5′ TCTTCATTTGGAGCATTCGG 3′ | (SEQ ID NO. 3) |
| and | |
| yst - 2 (R) | |
| 5′ ATTGCAACATACATCGCAGC 3′ | (SEQ ID NO. 4) |
| for detecting heat stable enterotoxin | |
| in Yersinia enterocolitica. | |

(b) a use for the detection of Staphylococcus aureus and Yersinia enterocolitica using the primers specific for enterotoxin A gene in Staphylococcus aureus and heat stable enterotoxin in Yersinia enterocolitica in a mixed microflora, (Please refer FIG. 3)
(c) preparing the food matrices for detecting Staphylococcus aureus and Yersinia enterocolitica in milk, ice cream and fruit juice,
(d) extracting the template DNA from Staphylococcus aureus and Yersinia enterocolitica, respectively, in milk, ice cream and fruit juice may be achieved using diethyl ether : chloroform in the ratio of 1:1-1:3, urea 1.5-3.5 M and sodium dodecyl sulphate, 0.5-2%.
(e) preparing the PCR reaction mixture in a total volume of 25 μl may consist of Tris HCl, 8- 12 mM; KCl, 45-55 mM; MgCl₂, 0.5-3.0 mM; gelatin, 0.005-0.02%; individual deoxynucleoside triphosphates, 150-300 μM; each specific primer, 30-60 picomoles; Taq DNA polymerase, 0.5-2.0 units and template DNA, 1-3 μl.
(f) amplifying the target genes for the detection of Staphylococcus aureus and Yersinia enterocolitica, respectively, may be effected from an initial denaturation at 90-98° C. for 2-8 min, amplification cycles of 28-40, each cycle with a denaturation at 90-98° C. for 40-70 seconds, annealing at 50-60° C. for 40-80 seconds and an extension at 68-76° C. for 45-75 seconds and final extension at 68-76° C. for 4-12 min
(g) analyzing the PCR product may be achieved in 1.2-1.8% agarose gel electrophoresis, visualization of the PCR product by staining with 0.5 μg/ml ethidium bromide and observed in a UV transilluminator.
(h) Detecting the minimum number of cells of Staphylococcus aureus and Yersinia enterocolitica, respectively, may be effected in a food matrix by PCR indicating high sensitivity.

In a preferred embodiment of the present invention, effective amplification of enterotoxin A and heat stable enterotoxin genes may be effected from an initial denaturation at 93-95° C. for 4-6 min, amplification cycles of 32-38, each cycle with a denaturation at 93 -95° C. for 55-65 seconds, annealing at 53-56° C. for 55-65 seconds and an extension at 70-74° C. for 55-65 seconds and final extension at 55-65° C. for 6-10 min

In another preferred embodiment of the present invention, the PCR use may detect 1 to 10⁶ cells of Staphylococcus aureus and Yersinia enterocolitica directly in foods.

In yet another embodiment of the present invention, the instant patent relates to an improved PCR use for the detection of Staphylococcus aureus and Yersinia enterocolitica in foods. Polymerase chain reaction use was used to selectively amplify enterotoxin A gene in Staphylococcus aureus and heat stable enterotoxin in Yersinia enterocolitica. Milk, ice cream and fruit juice samples were spiked with varying cell numbers of Staphylococcus aureus and Yersinia enterocolitica, individually ranging from 1 to 1,000,000. Protocols for extraction of template DNA from Staphylococcus aureus and Yersinia enterocolitica present in food matrix were standardized using detergents and organic solvents. The PCR reaction mixture and amplification conditions were optimized for the specific amplification. Visualization of PCR products revealed that by the use followed, it is possible to detect cell numbers ranging from 1 to 1,000,000 in milk, ice cream and fruit juice samples.

The novelty of this use is the use of the designed primers for the direct detection of Staphylococcus aureus and Yersinia enterocolitica in food systems by PCR. Besides, this use can detect enterotoxigenic/pathogenic strains of Staphylococcus aureus and Yersinia enterocolitica. The use is rapid and sensitive making it possible to detect even 1 cell in a food matrix overcoming any steps of enrichment.

In still another embodiment of the present invention, the main object of the present invention is to provide an improved use for the detection of Staphylococcus aureus and Yersinia enterocolitica in foods. The process of the present invention uses a primer designed for a conserved region of a specific gene in the target organisms, Staphylococcus aureus and Yersinia enterocolitica. The present invention provides a simple and effective use for the preparation of template DNA (Deoxyribo Nucleic Acid) of the organism directly from the foods. The use also uses PCR conditions specific for the detection of target genes in the respective organisms and detects very low numbers of target organism in the food systems, making the use very sensitive.

The invention of instant Application is further illustrated by the following examples which should not, however be construed to limit the scope of the invention.

### EXAMPLE 1

Oligonucleotide primers for enterotoxin A gene of Staphylococcus aureus were designed based on the gene sequence (M 18970) using the software programme Primer 3.0 This primer set amplifies a 301 base pair (bp) fragment of the gene, the sequence of which is given below. Sterilization of media and other solutions was achieved by autoclaving for 20 min at 121° C.

(SEQ ID NO. 1)

entA - 1 (F) 5′GGTAGGGAGAAAAGCGAAGA 3′

(SEQ ID NO. 2)

entA - 2 (R) 5′TACGACCCGCACATTGATAA 3′

Aliquots in 100 μl of a standard strain of Staphylococcus aureus FRI 722 was inoculated into sterile 10 ml brain heart infusion (BHI) broth and incubated for 18 h at 37° C. in a shaker incubator with 140 rpm. Cells were harvested by centrifugation at 10,000 rpm for 10 min at 4° C. The cells were suspended in 10 ml sterile 0.85% saline to get a cell concentration of 10⁹ colony forming units per millilitre (CFU/ml). From this stock, serial dilutions in 9-ml sterile 0.85% saline were carried out to achieve cell concentrations ranging from 10⁸ to 10¹ CFU/ml. The individual dilutions were used for spiking into individual food samples.

Twenty millilitres of pasteurized milk, ice cream and fruit juice, individually were taken in a sterile screw capped tube of 25×125 mm dimension and used as samples for the test. In individual 1.5 ml sterile microcentrifuge tube, 0.4 ml of the above individual food sample was mixed with 0.4 ml of 0.85% saline suspension of Staphylococcus aureus to attain a final cell concentration ranging from of 10⁶, 10⁵, 10⁴, 10³, 10², 10¹ and 10⁰ CFU/ml. To each tube was added 0.25 ml each of diethyl ether and chloroform were added to the samples and vortexed for 30 seconds. The samples were centrifuged at 10,000 rpm for 15 min at 25° C. The aqueous phase was transferred to a fresh 1.5 ml sterile microcentrifuge tube and 0.5 ml of 6M urea and 0.1 ml of 10% sodium dodecyl sulphate were added.

The samples were incubated at 37° C. for 20 min and then centrifuged 10,000 rpm for 15 min at 25° C. The supernatant was discarded and 0.1 ml of 0.2N NaOH was added to the samples and incubated at 37° C. for 10 min. DNA was precipitated by adding 1.0 ml of chilled absolute ethanol and 0.1 ml of 3M sodium acetate (pH 4.8) and holding the samples at −20° C. for 2 h. Samples were centrifuged at 10,000 rpm for 15 min at 4° C. The supernatant was discarded and excess salt in the DNA preparation was removed by adding 1.0 ml of chilled 70% ethanol and centrifuging the samples at 10,000 rpm for 15 min at 4° C. The supernatant was discarded and the DNA pellet was air-dried and resuspended in 15μl of sterile ultrafiltered water.

Amplification was performed in a total reaction volume of 25 μl which contained 2 μl of the DNA preparation from milk samples. The reaction mixture consisted of 1X PCR buffer (10 mM Tris HCl, pH 9.0, 50 mM KCl, 1.5 mM MgCl₂, 0.01% gelatin), 200 μM of each deoxynucleoside triphosphate, 50 picomoles of each primer and 1.0 unit of Taq DNA polymerase. Template DNAs were initially denatured at 94° C. for 5 min. Subsequently, a total of 35 amplification cycles were carried out in a programmable thermocycler. Each cycle consisted of denaturation for 1 min at 94° C., primer annealing for 1 min at 55° C. and extension for 1 min at 72° C. The last cycle was followed by a final extension at 72° C. for 8 min.

PCR products were analysed by agarose gel electrophoresis. Aliquots of 10 μl PCR products were mixed with 2.0 μl of loading dye and loaded onto 1.5% agarose gel and subjected to electrophoresis for 2 h at 120 volts in 1X TAE buffer. Gel was stained with ethidium bromide (0.5 μg/ml), destained with distilled water and examined on a UV transilluminator. A 100-bp ladder was used as molecular size marker. The amplification profile in the gel was documented in a CCD-camera based Gel Documentation System.

The specific amplicons of 301 bp for enterotoxin A gene were observed when PCR was performed with individual food samples containing Staphylococcus aureus cells ranging from 1 to 1,000,000.

### EXAMPLE 2

Oligonucleotide primers for heat stable enterotoxin gene of Yersinia enterocolitica were designed based on the gene sequence (X 65999) using the software programme Primer 3.0 This primer set amplifies a 159 base pair (bp) fragment of the gene, the sequence of which is given below. Sterilization of media and other solutions was achieved by autoclaving for 20 min at 121° C.

(SEQ ID NO.3)

yst - 1 (F) 5′TCTTCATTTGGAGCATTCGG 3′

(SEQ ID NO.4)

yst - 2 (R) 5′ATTGCAACATACATCGCAGC 3′

Aliquots in 100, μl of a standard strain of Yersinia enterocolitica MTCC 859 was inoculated into sterile 10 ml brain heart infusion (BHI) broth and incubated for 18 h at 32° C. in a shaker incubator with 140 rpm. Cells were harvested by centrifugation at 10,000 rpm for 10 min at 4° C. The cells were suspended in 10 ml sterile 0.85% saline to get a cell concentration of 10⁹ colony forming units per millilitre (CFU/ml). From this stock, serial dilutions in 9 ml sterile 0.85% saline were carried out to achieve cell concentrations ranging from 10⁸ to 10¹ CFU/ml. The individual dilutions were used for spiking into individual food samples.

Twenty milliliters of pasteurized milk, ice cream and fruit juice, individually were taken in a sterile screw capped tube of 25×125 mm dimension and used as samples for the test. In individual 1.5 ml sterile microcentrifuge tube, 0.4 ml of the above individual food sample was mixed with 0.4 ml of 0.85% saline suspension of Yersinia enterocolitica to attain a final cell concentration ranging from of 10⁶, 10⁵, 10⁴, 10³, 10², 10¹ and 10⁰ CFU/ml. To each tube was added 0.25 ml each of diethyl ether and chloroform were added to the samples and vortexed for 30 seconds. The samples were centrifuged at 10,000 rpm for 15 min at 25° C. The aqueous phase was transferred to a fresh 1.5 ml sterile microcentrifuge tube and 0.5 ml of 6M urea and 0.1 ml of 10% sodium dodecyl sulphate were added.

The samples were incubated at 37° C. for 20 min and then centrifuged 10,000 rpm for 15 min at 25° C. The supernatant was discarded and 0.1 ml of 0.2N NaOH was added to the samples and incubated at 37° C. for 10 min. DNA was precipitated by adding 1.0 ml of chilled absolute ethanol and 0.1 ml of 3M sodium acetate (pH 4.8) and holding the samples at −20° C. for 2 h. Samples were centrifuged at 10,000 rpm for 15 min at 4° C. The supernatant was discarded and excess salt in the DNA preparation was removed by adding 1.0 ml of chilled 70% ethanol and centrifuging the samples at 10,000 rpm for 15 min at 4° C. The supernatant was discarded and the DNA pellet was air-dried and resuspended in 15 μl of sterile ultrafiltered water.

Amplification was performed in a total reaction volume of 25 μl which contained 2 μl of the DNA preparation from milk samples. The reaction mixture consisted of 1X PCR buffer (10 mM Tris HCl, pH 9.0, 50 mM KCl, 1.5 mM MgCl₂, 0.01% gelatin), 200 μM of each deoxynucleoside triphosphate, 50 picomoles of each primer and 1.0 unit of Taq DNA polymerase. Template DNAs were initially denatured at 94° C. for 5 min. Subsequently, a total of 35 amplification cycles were carried out in a programmable thermocycler. Each cycle consisted of denaturation for 1 min at 94° C., primer annealing for 1 min at 55° C. and extension for 1 min at 72° C. The last cycle was followed by a final extension at 72° C. for 8 min.

PCR products were analysed by agarose gel electrophoresis. Aliquots of 10 μl PCR products were mixed with 2.0 μl of loading dye and loaded onto 1.5-% agarose gel and subjected to electrophoresis for 2 h at 120 volts in 1X TAE buffer. Gel was stained with ethidium bromide (0.5 μg/ml), destained with distilled water and examined on a UV transilluminator. A 100 bp ladder was used as molecular size marker. The amplification profile in the gel was documented in a CCD-camera based Gel Documentation System.

The specific amplicons of 159 bp for heat stable enterotoxin were observed when PCR was performed with individual food samples containing Yersinia enterocolitica cells ranging from 1 to 1,000,000.

A. The Details of the DNA Sequence of Enterotoxin A Gene of S. aureus Selected from the Database is as Follows

```
M18970. S.aureus enteroto...[gi:153120] Related Sequences, Protein, PubMed, Taxonomy
LOCUS STATOXAA 774 bp DNA linear BCT 26-APR-1993
DEFINITION S.aureus enterotoxin A (entA) gene, complete cds.
ACCESSION M18970
VERSION M18970.1 GI:153120
KEYWORDS enterotoxin.
SOURCE S.aureus (strain FRI337) DNA, clones pMJB[9,38].
ORGANISM Staphylococcus aureus
        Bacteria; Firmicutes; Bacillus/Clostridium group; Bacillales;
        Staphylococcus.
REFERENCE 1 (bases 1 to 774)
AUTHORS Betley,M.J. and Mekalanos,J.J.
TITLE Nucleotide sequence of the type A staphylococcal enterotoxin gene
JOURNAL J.Bacteriol. 170, 34-41 (1988)
MEDLINE 88086892
FEATURES Location/Qualifiers
    source 1..774
                /organism=“Staphylococcus aureus”
                /db_xref=“taxon:1280”
    sig_peptide 1..72
                /note=“staphylococcal enterotoxin A signal peptide”
    CDS 1..774
                /note=“staphylococcal enterotoxin A precursor”
                /codon_start=1
                /transl_table=11
                /protein_id=“AAA26681.1”
                /db_xref=“GI:153121”
/translation=“MKKTAFTLLLFIALTLTTSPLVNGSEKSEEINEKDLRKKSELQGTALG
NLKQIYYYNEKAKTENKESHDQFLQHTILFKGFFTDHSWYNDLLVDFDSKDIVDK
YKGKKVDLYGAYYGYQCAGGTPNKTACMYGGVTLHDNNRLTEEKKVPINLWLD
GKQNTVPLETVKTNKKNVTVQELDLQARRYLQEKYNLYNSDVFDGKVQRGLIVF
HTSTEPSVNYDLFGAQGQYSNTLLRIYRDNKTINSENMHIDIYLYTS”
    mat_peptide 73..771
                    /product=“staphylococcal enterotoxin A”
BASECOUNT 299 a 97 c 144 g 234 t
ORIGIN 47 bp upstream of Hincii site.
      1 atgaaaaaaa cagcatttac attactttta ttcattgccc taacgttgac aacaagtcca
      61 cttgtaaatg gtagcgagaa aagcgaagaa ataaatgaaa aagatttgcg aaaaaagtct
      121 gaattgcagg gaacagcttt aggcaatctt aaacaaatct attattacaa tgaaaaagct
      181 aaaactgaaa ataaagagag tcacgatcaa tttttacagc atactatatt gtttaaaggc
      241 ttttttacag atcattcgtg gtataacgat ttattagtag attttgattc aaaggatatt
      301 gttgataaat ataaagggaa aaaagtagac ttgtatggtg cttattatgg ttatcaatgt
      361 gcgggtggta caccaaacaa aacagcttgt atgtatggtg gtgtaacgtt acatgataat
      421 aatcgattga ccgaagagaa aaaagtgccg atcaatttat ggctagacgg taaacaaaat
      481 acagtacctt tggaaacggt taaaacgaat aagaaaaatg taactgttca ggagttggat
      541 cttcaagcaa gacgttattt acaggaaaaa tataatttat ataactctga tgtttttgat
      601 gggaaggttc agaggggatt aatcgtgttt catacttcta cagaaccttc ggttaattac
      661 gatttatttg gtgctcaagg acagtattca aatacactat taagaatata tagagataat
      721 aaaacgatta actctgaaaa catgcatatt gatatatatt tatatacaag ttaa
```

The sequence of the conserved region of enterotoxin A gene of S. aureus selected from the above shown sequence is given below and the regions flanked by the forward and reverse primers mentioned in the patent application are indicated in bold letters. The primers have been designed to achieve high sensitivity of detection in food systems.

ggtagcgagaa aagcgaagaa ataaatgaaa aagatttgcg
aaaaaagtct
gaattgcagg gaacagcttt aggcaatctt aaacaaatct
attattacaa tgaaaaagct
aaaactgaaa ataaagagag tcacgatcaa tttttacagc
atactatatt gtttaaaggc
ttttttacag atcattcgtg gtataacgat ttattagtag
attttgattc aaaggatatt
gttgataaat ataaagggaa aaaagtagac ttgtatggtg
cttattatgg ttatcaatgt
gcgggtggta

B. The Details of the DNA Sequence of Heat Stable Enterotoxin Gene of Y. enterocolitica Selected from the Database is as Follows

```
X65999. Y.enterocolitica ...[gi:48611] Related Sequences, Protein, Taxonomy
LOCUS YEYSTG 216 bp DNA linear BCT 06-OCT-1992
DEFINITION Y.enterocolitica yst gene for enterotoxin.
ACCESSION X65999
VERSION X65999.1 GI:48611
KEYWORDS enterotoxin.
SOURCE Yersinia enterocolitica.
ORGANISM Yersinia enterocolitica
        Bacteria; Proteobacteria; gamma subdivision; Enterobacteriaceae;
        Yersinia.
REFERENCE 1 (bases 1 to 216)
AUTHORS  Stackebrandt,E.
TITLE  Direct Submission
JOURNAL  Submitted (06-MAY-1992) E. Stackebrandt, Dept of Microbiology,
          University of Queensland, St.Lucia, Qld, AUSTRALIA 4072
REFERENCE 2 (bases 1 to 216)
AUTHORS Ibrahim,A., Liesack,W., Pike,S. and Stackebrandt,E.
TITLE The Polymerase chain reaction: an epidemiological tool to
          differentiate between two clusters of pathogenic yersinia
          enterocolitica strains
JOURNAL FEMS Microbiol. Lett. 97, 63-66 (1992)
FEATURES Location/Qualifiers
    source 1..216
                /organism=“Yersinia enterocolitica”
                /strain=“serotype 0:8”
                /db_xref=“taxon:630”
    gene 1..216
                /gene=“yst”
    CDS 1..216
                /gene=“yst”
                /codon_start=1
                /transl_table=11
                /product=“enterotoxin”
                /protein_id=“CAA46801.1”
                /db_xref=“GI:48612”
                /db_xrefr=“SWISS-PROT:P07593”
/translation=“MKKIVFVLVLMLSSFGAFGQETVSGQFSDALSTPITAEVYKQAC
                          DPPLPPAEVSSDWDCCDVCCNPACAGC”
BASECOUNT 52 a 44 c 56 g 64 t
ORIGIN
      1 atgaaaaaga tagtttttgt tcttgtgtta atgctgtctt catttggagc attcggccaa
      61 gaaacagttt cagggcagtt cagtgatgca ttatcgacac caataaccgc tgaggtatac
      121 aagcaagctt gtgatcctcc gctgccacca gccgaagtca gtagtgattg ggattgctgc
      181 gatgtatgtt gcaatcctgc ctgtgcgggt tgctag
```

The sequence of the conserved region of heat stable enterotoxin gene of Y. enterocolitica selected from the above shown sequence is given below and the regions flanked by the forward and reverse primers mentioned in the patent application are indicated in bold letters. The primers have been designed to achieve high sensitivity of detection in food systems.

tctt catttggagc attcggccaa gaaacagttt cagggcagtt cagtgatgca ttatcgacac caataaccgc
tgaggtatac aagcaagctt gtgatcctcc gctgccacca gccgaagtca gtagtgattg ggattgctgc
gatgtatgtt gcaat

1. Oligonucleotide primers of SEQ ID Nos. 1, 2, 3, and 4.

2. Primers as claimed in claim 1, wherein said primers are of size 20 nucleotides.

3. Primers as claimed in claim 1, wherein primers of SEQ ID Nos. 1, and 2 target enterotoxin A gene (entA) of food poisoning bacterial species Staphylococcus aureus.

4. Primers as claimed in claim 1, wherein primers of SEQ ID Nos. 3, and 4 target heat stable enterotoxin gene (yst) of Yersinia enterocolitica.

5. Primers as claimed in claim 1, wherein primer of SEQ ID Nos. 1 and 3 are forward primers.

6. Primers as claimed in claim 1, wherein primer of SEQ ID No. 2 and 4 are reverse primers.

7. A use of preparing primers of SEQ ID Nos. 1-4 of claim 1, said use comprising steps of:
(a) identifying conserved sequence of entA, and yst genes of bacterial strains Staphylococcus aureus and Yersinia enterocolitica respectively.
(b) generating primers using software programme.

8. A use as claimed in claim 7, wherein conserved sequence of entA gene is located in a region between 70-370.

9. A use as claimed in claim 7, wherein conserved sequence of yst gene is located in a region between 37-195.

10. A use as claimed in claim 7, wherein software programme is Primer 3.0

11. A highly sensitive and quick use of simultaneously detecting food poisoning bacterial species staphylococcus aureus and/or Yersinia enterocolitica in food systems without prior enrichment using specific primers of SEQ ID Nos. 1 and 2, and/or 3 and 4 of claim 1, said use comprising:
(a) preparing food matrix,
(b) extracting total microbial DNA,
(c) amplifying profile of target gene by PCR using said primers,
(d) analyzing PCR product by gel-electrophoresis, and
(e) detecting said bacterial strain,

12. A use as claimed in claim 11, wherein food system is selected from a group comprising milk, fruit juices, and ice creams.

13. A use as claimed in claim 11, wherein extracting. DNA by using extraction mixture comprising diethyl ether, chloroform, urea, and sodium dodecyl sulphate (SDS).

14. A use as claimed in claim 13, wherein diethyl ether and chloroform are in the ratio ranging between 1:1-1:5.

15. A use as claimed in claim 13, wherein concentration of urea is ranging between 1.0 to 4.5 M.

16. A use as claimed in claim 13, wherein concentration of SDS is ranging between 0.3-3.0%.

17. A use as claimed in claim 11, wherein PCR reaction mixture is comprising Tris Hydrochloric acid (Tris HCl) ranging between 6-15 mM, Potassium Chloride (KCl) ranging between 40-60 mM, Magnesium Chloride (MgCl₂) ranging between 0.3-5.0 mM, gelatin ranging between 0.002-0.05%, individual deoxynucleotide triphosphates ranging between 100-500 μM, each specific primer of claim 1, Taq DNA polymerase ranging between 0.3-5.0 units, template DNA ranging between 0.02-3.0%.

18. A use as claimed in claim 11, wherein denaturing DNA in PCR at temperature ranging between 90-98° C. for time period ranging between 1-10 minutes.

19. A use as claimed in claim 18, wherein denaturing DNA in PCR at temperature preferably ranging between 93-95° C. for time period ranging between 4-6 minutes.

20. A use as claimed in claim 11, wherein running PCR with amplification cycles ranging between 25-45 cycles.

21. A use as claimed in claim 20, wherein running PCR with amplification cycles preferably ranging between 32-38 cycles.

22. A use as claimed in claim 11, wherein denaturation temperature at each cycle is ranging between 90-98° C. for time period ranging between 30-80 seconds.

23. A use as claimed in claim 22, wherein denaturation temperature at each cycle is preferably ranging between 93-95° C. for time period ranging between 55-65 seconds.

24. A use as claimed in claim 11, wherein annealing DNA in PCR at temperature ranging between 40-65° C. for time period ranging between 30-90 seconds.

25. A use as claimed in claim 24, wherein annealing DNA in PCR at temperature preferably ranging between 53-56° C. for time period ranging between 55-65 seconds.

26. A use as claimed in claim 11, wherein extension at PCR is at temperature ranging between 68-76° C. for time period ranging between 40-80 seconds.

27. A use as claimed in claim 26, wherein extension at PCR is at temperature preferably ranging between 70-74° C. for time period ranging between 55-65 seconds.

28. A use as claimed in claim 11, wherein final extension at PCR is at temperature ranging between 68-76° C. for time period ranging between 2-15 minutes.

29. A use as claimed in claim 28, wherein final extension at PCR is at temperature preferably ranging between 55-65° C. for time period ranging between 6-10 minutes.

30. A use as claimed in claim 11, wherein gel electrophoresis is run on agarose gel.

31. A use as claimed in claim 30, wherein concentration of agarose gel is ranging between 1.0-2.0%.

32. A use as claimed in claim 31, wherein staining agarose gel with Ethidium bromide at a concentration ranging between 0.2-1.0 μg/ml.

33. A use as claimed in claim 32, wherein stained gel is observed under UV transilluminator.

34. A use as claimed in claim 11, wherein said use is used to detect said bacterial strains in quantity as low as one cell.

35. A use as claimed in claim 11, wherein said use help prevent food poisoning outbreak.

36. A use as claimed in claim 11, wherein said use is a direct use of detecting bacterial strain.


SEQUENCE LISTING (10 sequences)

SEQ ID NO: 1 - DNA, 20 bp, Artificial Sequence
  Description of Artificial Sequence Synthetic primer
ggtagcgagaaaagcgaaga

SEQ ID NO: 2 - DNA, 20 bp, Artificial Sequence
  Description of Artificial Sequence Synthetic primer
taccacccgcacattgataa

SEQ ID NO: 3 - DNA, 20 bp, Artificial Sequence
  Description of Artificial Sequence Synthetic primer
tcttcatttggagcattcgg

SEQ ID NO: 4 - DNA, 20 bp, Artificial Sequence
  Description of Artificial Sequence Synthetic primer
attgcaacatacatcgcagc

SEQ ID NO: 5 - protein, 257 aa, Staphylococcus aureus
MKKTAFTLLLFIALTLTTSPLVNGSEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIDIYLYTS

SEQ ID NO: 6 - DNA, 774 bp, Staphylococcus aureus
atgaaaaaaacagcatttacattacttttattcattgccctaacgttgacaacaagtccacttgtaaatggtagcgagaaaagcgaagaaataaatgaaaaagatttgcgaaaaaagtctgaattgcagggaacagctttaggcaatcttaaacaaatctattattacaatgaaaaagctaaaactgaaaataaagagagtcacgatcaatttttacagcatactatattgtttaaaggcttttttacagatcattcgtggtataacgatttattagtagattttgattcaaaggatattgttgataaatataaagggaaaaaagtagacttgtatggtgcttattatggttatcaatgtgcgggtggtacaccaaacaaaacagcttgtatgtatggtggtgtaacgttacatgataataatcgattgaccgaagagaaaaaagtgccgatcaatttatggctagacggtaaacaaaatacagtacctttggaaacggttaaaacgaataagaaaaatgtaactgttcaggagttggatcttcaagcaagacgttatttacaggaaaaatataatttatataactctgatgtttttgatgggaaggttcagaggggattaatcgtgtttcatacttctacagaaccttcggttaattacgatttatttggtgctcaaggacagtattcaaatacactattaagaatatatagagataataaaacgattaactctgaaaacatgcatattgatatatatttatatacaagttaa

SEQ ID NO: 7 - DNA, 300 bp, Staphylococcus aureus
ggtagcgagaaaagcgaagaaataaatgaaaaagatttgcgaaaaaagtctgaattgcagggaacagctttaggcaatcttaaacaaatctattattacaatgaaaagctaaaactgaaaataaagagagtcacgatcaatttttacagcatactatattgtttaaaggcttttttacagatcattcgtggtataacgatttattagtagattttgattcaaaggatattgttgataaatataaagggaaaaaagtagacttgtatggtgcttattatggttatcaatgtgcgggtggta

SEQ ID NO: 8 - protein, 71 aa, Yersinia enterocolitica
MKKIVFVLVLMLSSFGAFGQETVSGQFSDALSTPITAEVYKQACDPPLPPAEVSSDWDCCDVCCNPACAGC

SEQ ID NO: 9 - DNA, 216 bp, Yersinia enterocolitica
atgaaaaagatagtttttgttcttgtgttaatgctgtcttcatttggagcattcggccaagaaacagtttcagggcagttcagtgatgcattatcgacaccaataaccgctgaggtatacaagcaagcttgtgatcctccgctgccaccagccgaagtcagtagtgattgggattgctgcgatgtatgttgcaatcctgcctgtgcgggttgctag

SEQ ID NO: 10 - DNA, 159 bp, Yersinia enterocolitica
tcttcatttggagcattcggccaagaaacagtttcagggcagttcagtgatgcattatcgacaccaataaccgctgaggtatacaagcaagcttgtgatcctccgctgccaccagccgaagtcagtagtgattgggattgctgcgatgtatgttgcaat